(19) United States
(12) Patent Application Publication
(10) Pub. No.: US 2005/0065454 A1
(43) Pub. Date: Mar. 24, 2005

(54) Non-evacuated blood collection tube

(75) Inventor: Dimitrios Manoussakis, Wyckoff, NJ (US)

Correspondence Address: WEBB ZIESENHEIM LOGSDON ORKIN & HANSON, P.C., 700 KOPPERS BUILDING, 436 SEVENTH AVENUE, PITTSBURGH, PA, 15219, US

(73) Assignee: Becton, Dickinson And Company

(21) Appl. No.: 10/667,920

(22) Filed: Sep. 22, 2003

(51) Int. Cl. A61B 5/00

(52) U.S. Cl. 600/576

(57) ABSTRACT

A specimen collection container for collecting a biological sample such as blood which includes a non-evacuated collection tube. The tube includes a vent which is adapted for displacement of air from within the interior chamber of the collection tube to the external environment during collection of a blood sample therein. Desirably, the vent is air permeable and liquid impermeable, and seals upon contact with a liquid sample so as to become air impermeable.

### BACKGROUND OF THE INVENTION

1. Field of the Invention

The present invention relates to a blood collection tube. More particularly, the present invention relates to a non-evacuated blood collection tube for use by medical professionals in the collection of fluid samples from humans.

2. Description of Related Art

Blood collection containers are well-known in the medical arts. They are used to store a sample of blood obtained by a phlebotomist from a patient until the blood is ready to be tested or used for other purposes. Currently, most blood collection containers on the market are evacuated. That is, a vacuum exists within the interior of a collection tube and a piercible stopper is located at the injection end of the collection tube. The vacuum is used to provide a draw on the blood received from a patient into the interior of the collection tube. In an ideal situation, once the piercible stopper end of a typical blood collection tube is pierced via a needle in a blood collection set, blood will be drawn into the interior of the collection tube until the vacuum is exhausted, or more likely, until the tube is removed from the needle of the blood collection set.

Ideal situations in the practice of blood collection do not always occur. Evacuated blood collection tubes need to maintain the vacuum inside the collection tube in order for the blood sampling to work. Once the vacuum is gone, the tube will no longer function as promised. Moreover, it is difficult to control the rate of draw by means of an evacuated tube, other than with partial draw tubes. This uncontrollability can give rise to vein collapse if the vacuum draw is too strong for a given patient's veins, especially a concern in the elderly and infants or severely ill patients.

Blood extraction devices for small sample volumes within sample tubules has been proposed, for example in U.S. Pat. No. 5,115,817 to Sarstedt. This patent discloses a sample tubule including an inner tube therein which is closed through a cap, and which includes a hermetic seal provided through a self-closing membrane. During blood extraction, a needle pierces through the membrane and blood is collected within the inner tube. After extraction, the cap is unscrewed, and air vents within the sample tubule vent air such that the inner tube can be removed from the sample tubule while the blood is conveyed at the bottom of the sample tubule. Such a blood extraction device does not provide for displacement of air within the sample tubule during the extraction, and therefore provides for limited sampling capability. Also, the device of this patent includes open vent bores for transfer of the blood into the sample tubule which pose a risk for contamination, and requires a complicated procedure for removal of the inner tube to transfer the blood to the sample tubule.

Therefore, a need exists for a non-evacuated blood collection tube for use in medical sampling procedures which is reliable, safe, convenient, and cost-effective.

### SUMMARY OF THE INVENTION

The present invention is directed to a non-evacuated specimen collection container which is capable of collecting a blood sample from a patient based on the physiological venous pressure of the patient, without the need for any internal vacuum within the collection container. The collection container includes a non-evacuated collection tube having a tubular wall extending between an open top end and a bottom end defining an interior chamber, a piercible closure sealing the open top end of the collection tube, and a vent adapted for displacement of air from within the interior chamber of the collection tube to an exterior of the collection tube during collection of a liquid sample within the interior chamber of the collection tube. The vent is adapted for maintaining the interior chamber at ambient pressure prior to collection of a sample, and may be a two-way vent. Desirably, the vent is adapted to seal upon contact with the liquid sample so as to prevent further displacement of air into or out from the tube.

Desirably, the tube is open at both the top and bottom ends, with a piercible stopper sealing the top end and a hybrid stopper including a venting filter sealing the bottom end. In a further embodiment, a collapsible bag may be removably mated with the open top end of the collection tube, with the bag positioned in the interior chamber of the tube. In this manner, the blood sample may be collected within the collapsible bag, with the bag expanding upon filling under venous pressure.

In a further embodiment, the present invention is directed to a method for collecting a biological sample. In the method, a non-evacuated collection tube including a vent adapted for displacement of air from within the tube is provided with a piercible closure for accessing the interior of the collection tube. A biological sample is transferred through the piercible closure, such as by way of a needle cannula extending from a patient's vein and piercing through the piercible closure, and is forced into the interior chamber based on the venous pressure of the patient, such that any air present within the interior of the collection tube will vent to the exterior environment through the vent. Desirably, the vent is adapted to seal upon contact with the biological sample so as to prevent further displacement of air.

### DESCRIPTION OF THE DRAWINGS

FIG. 1 is a perspective view of a collection tube in accordance with the present invention;

FIGS. 2 and 3 are exploded perspective views of the collection tube of FIG. 1;

FIG. 4 is a side cross-sectional view of the collection tube of FIG. 1;

FIG. 5 is a perspective view of the collection tube of the present invention in use with a needle during a blood collection procedure;

FIG. 6 is a top perspective view of a collection tube in accordance with a further embodiment of the present invention;

FIG. 7 is a bottom perspective view of the collection tube of FIG. 6;

FIG. 8 is a side cross-sectional view of the collection tube of FIG. 6;

FIG. 9 is an exploded perspective view of a collection tube in a further embodiment;

FIG. 10 is a side cross-sectional view of the tube of FIG. 9;

FIG. 11 is a perspective view of a further embodiment in an empty state prior to collection of a sample;

FIG. 12 is a perspective view of the tube of FIG. 11 filled with a sample;

FIG. 13 is a side cross-sectional view of the tube of FIG. 11; and

FIG. 14 is a side cross-sectional view of a tube in yet a further embodiment.

### DETAILED DESCRIPTION

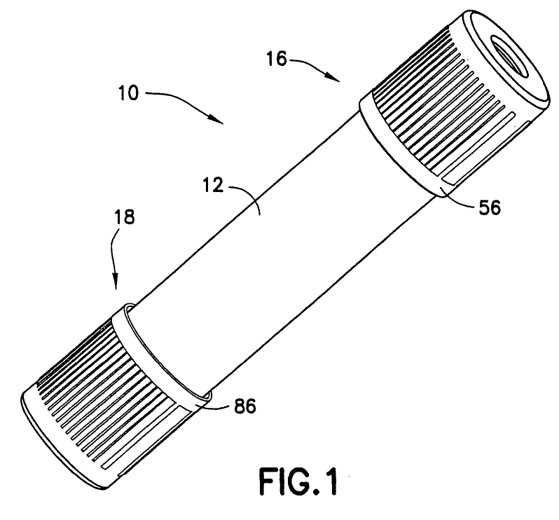

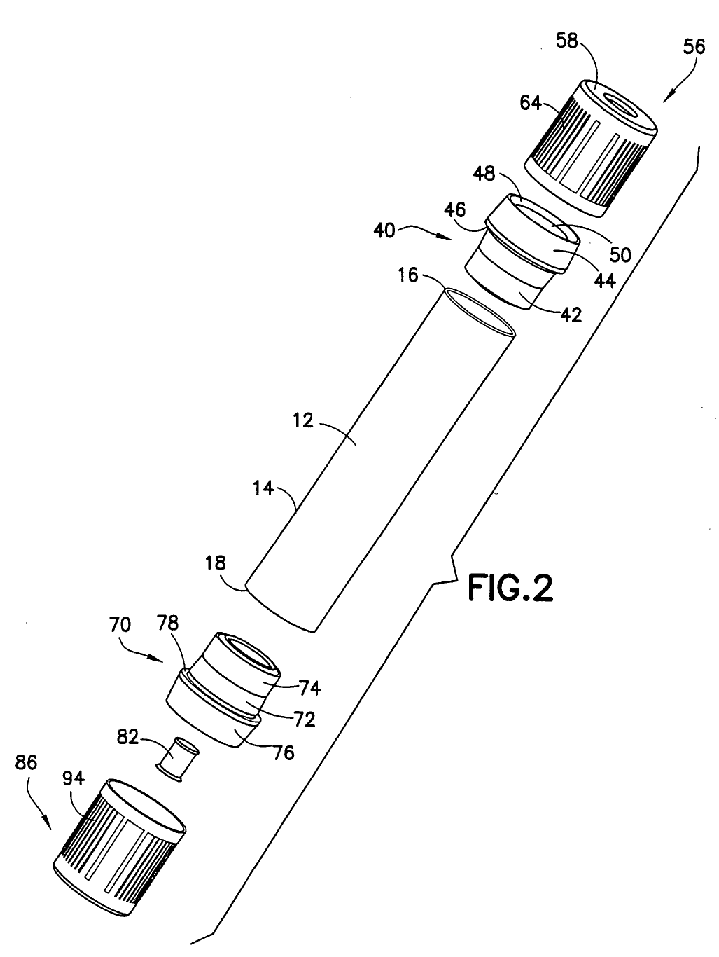

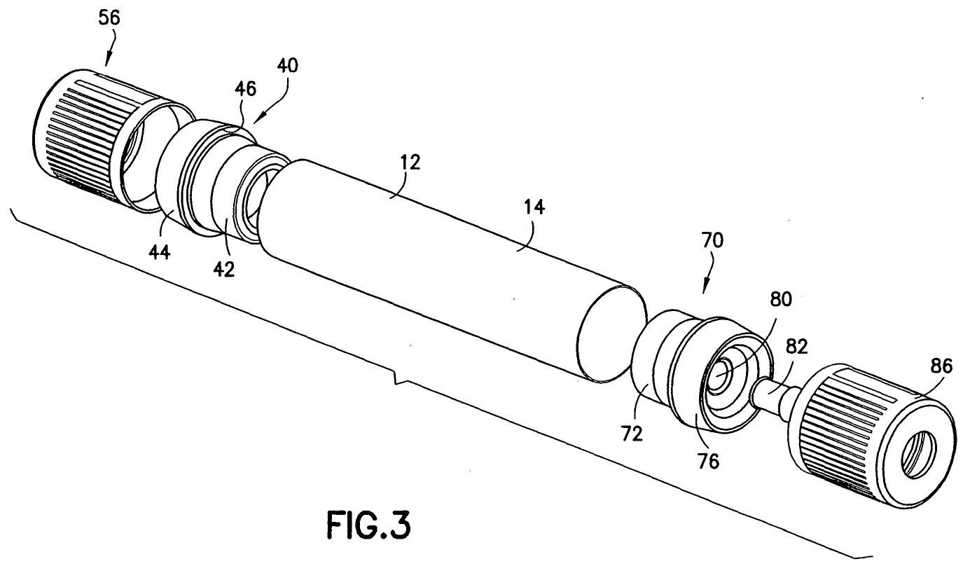

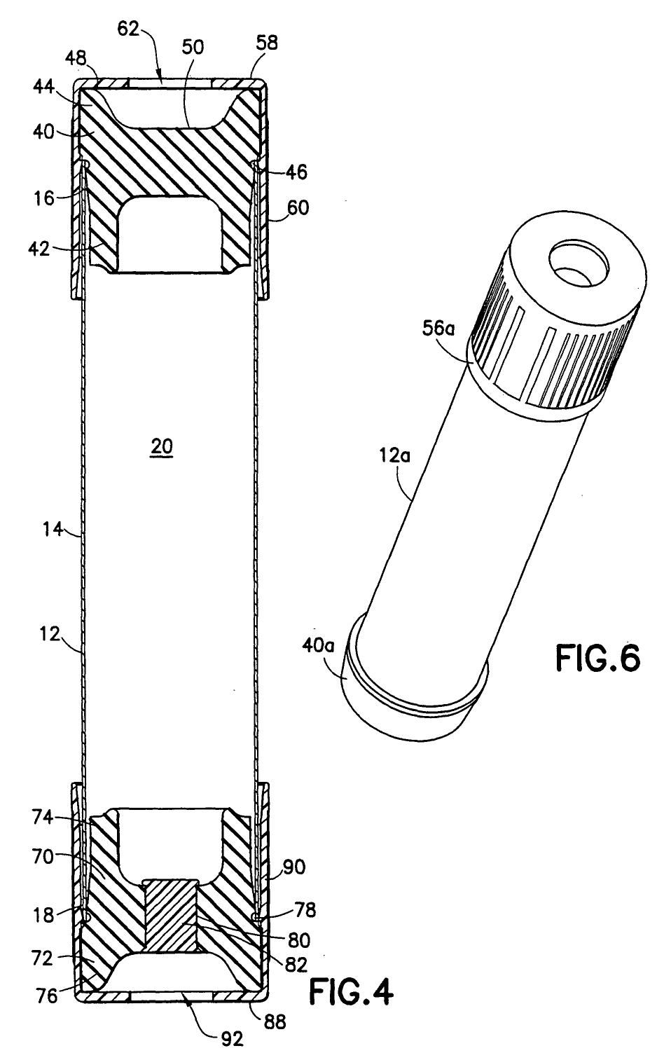

Referring to the drawings in which like reference characters refer to the like parts throughout the several views thereof. FIGS. 1-4 illustrate a specimen collection container in accordance with a preferred embodiment of the present invention and the related features. As shown in FIGS. 1-4, the specimen collection container 10 includes a tube 12, including a piercible closure 40 at one end and a vent such as hybrid stopper 70 extending from the opposing end thereof.

More particularly, tube 12 includes a generally cylindrical tubular wall 14 extending between a first end 16 at the top thereof and a second end 18 at the bottom thereof, defining an interior chamber 20 of tube 12. Tube 12 may be made out of any suitable material which is impermeable to liquid, preferably impermeable to gas and liquid, and is desirably made out of glass or molded plastic. Additionally, tube 12 may be constructed in any practical size for obtaining an appropriate biological sample. For example, tube 12 may be of a size similar to conventional large volume tubes, small volume tubes, or microcollection tubes, as is known in the art.

Tube 12 includes a piercible closure 40 sealingly covering first end 16 thereof. Piercible closure 40 is constructed of a suitable material capable of providing the open first end 16 of tube 12 with a liquid-tight seal, and capable of being punctured or pierced with an appropriate medical device, such as a needle cannula, for transfer of a biological sample into tube 12. Desirably, piercible closure 40 is constructed of an elastomeric material, such as rubber.

Piercible closure 40 may be mated with first end 16 of tube 12 through any mechanism, such as a snap-fit, a friction fit, a threaded connection, or the like. Desirably, piercible closure 40 is constructed of an elastomeric material and is mated with first end 16 in a friction-fit arrangement. For example, piercible closure 40 may include a depending portion 42 extending from a main portion 44, with depending portion 42 extending within the opening at first end 16 of tube 12 and into interior chamber 20 thereof. Piercible closure 40 includes an annular shoulder 46 which mates with first end 16 of tube 12. Further, piercible closure 40 has a top surface 48, which may include a depending recess 50 centrally molded or fabricated thereon. The outer diameter of depending portion 42 of piercible closure 40 is substantially the same as the inner diameter of interior chamber 20 of tube 12. Also, the outer diameter of main portion 44 at annular shoulder 46 of piercible closure 40 is greater than the inner diameter of interior chamber 20 of tube 12. In this manner, when piercible closure 40 is removably mated to first end 16 of tube 12 annular shoulder 46 rests on the edge of first end 16, and a gas- and liquid-tight seal is formed.

A cap element 56 may further be provided and mated with first end 16 of tube 12 about piercible closure 40. More particularly, cap element 56 may be provided including a front face 58 with an annular skirt 60 depending therefrom. Front face 58 of cap element 56 includes a central opening such as through hole 62 extending therethrough. Cap element 56 is desirably positioned over piercible closure 40 and is engaged with the outer surface of tube 12 at first end 16 thereof, such as through a friction-fit, a snap-fit, a threaded engagement or the like, or may be adhesively affixed or adhered thereto. Cap element 56 may desirably be constructed of a plastic or polymeric material, and desirably includes a series of ribs 64 for providing a tactile surface. Also, cap element 56 may be color-coded, providing the user with an identification of the contents of the tube 12, or the intended use or intended testing of the contents of the tube 12.

Tube 12 further includes a vent element at opposing second end 18 thereof. Such a vent element is a physical structure which is adapted for venting of air within interior chamber 20 of tube 12, while maintaining a closed environment, in particular a liquid-tight environment, within interior chamber 20 of tube 12. The vent element may be integrally formed with tube 12, or may be a separate element which is separately attached to tube 12.

Desirably, such a vent element is provided through a hybrid stopper 70 which includes an opening for accommodating a venting structure therein and maintaining the venting structure in fluid communication with the interior chamber 20 of tube 12. For example, hybrid stopper 70 may be mated with an open second end 18 through any mechanism, such as a snap-fit, a friction fit, a threaded connection, or the like, such as with piercible closure 40 mated at first end 16. Desirably, hybrid stopper 70 is constructed of an elastomeric material such as rubber, and includes a depending portion 74 extending from a main portion 76, with an annular shoulder 78 therebetween. Annular shoulder 78 mates with second end 18 of tube 12 such that depending portion 74 extends within the opening at second end 18 and into interior chamber 20 thereof. As with the piercible closure 40, the outer diameter of depending portion 74 of hybrid stopper 70 is substantially the same as the inner diameter of interior chamber 20 of tube 12, and the outer diameter of main portion 76 at annular shoulder 78 of hybrid stopper 70 is greater than the inner diameter of interior chamber 20 of tube 12. In this manner, when hybrid stopper 70 is removably mated to second end 18 of tube 12 annular shoulder 78 rests on the edge of second end 18, and a gas- and liquid-tight seal is formed.

Hybrid stopper 70 further includes a central opening 80 extending therethrough between opposing ends thereof. Central opening 80 provides a path for fluid communication between interior chamber 20 of tube 12 and the external environment when hybrid stopper 70 is sealingly positioned over open second end 18. A venting filter 82 is located within central opening 80, and is desirably affixed therein, for example through a suitable medical grade adhesive. Venting filter 82 permits air and other gases to flow from within internal chamber 20 to the external environment. Venting filter 82 may be a two-way vent, in which air is able to flow in both directions within and out of internal chamber 20, thereby facilitating adjustment in different barometric or ambient pressures. While the venting filter 82 permits air and gas to flow therethrough, it acts as a barrier for liquid flow in either direction. Also, once a liquid specimen such as blood contacts and/or saturates the venting filter 82, the vent will seal to any further gas flow therethrough. As such, no air will be able to flow in or out of the vent, even once the liquid specimen has been removed. This characteristic decreases the chances of contaminants being able to enter the tube and contact a liquid sample contained within the interior chamber of the tube. However, this characteristic necessitates care on the part of the phlebotomist in that the liquid sample should not contact the venting filter 82 prematurely.

Venting filter 82 may be made out of any suitable material which is permeable to gases (in particular air) and impermeable to liquids (in particular blood), and is desirably constructed of a polymeric material such as high density polyethylene or high density polypropylene. A particularly useful material is available from Porex Porous Products.

As with first end 16 of tube 12, a cap element 86 may further be provided and mated with second end 18 of tube 12 about hybrid stopper 70. More particularly, cap element 86 may be provided including a front face 88 with an annular skirt 90 depending therefrom. Front face 88 of cap element 86 includes a central opening such as through-hole 92 extending therethrough. Cap element 86 is desirably positioned over hybrid stopper 70 and is engaged with the outer surface of tube 12 at second end 18 thereof, such as through a friction-fit, a snap-fit, a threaded engagement or the like, or may be adhesively affixed or adhered thereto. Cap element 86 may desirably be constructed of a plastic or polymeric material, and desirably includes a series of ribs 94 for providing a tactile surface. Also, cap element 86 may be color-coded, desirably with the same color coding as cap element 56 at first end 16 of tube 12.

It is also contemplated that the arrangement of the components of the hybrid stopper may be reversed. For example, the venting filter may be constructed of a material which can provide an effective liquid-tight seal directly within the tubular wall 14 at open bottom end 18 of tube 12, without the need for any separate elastomeric material sealing against the tubular wall 14. In such an arrangement, a separate piercible insert member may be provided through the venting filter, for providing a mechanism for piercible access. Such an embodiment effectively provides for an annular venting filter around a piercible insert member. It is further contemplated that in such an embodiment, such a hybrid stopper including a piercible insert within an annular filter member forms a closure at one end of the tube, while the opposing end of the tube may be closed off through a separate stopper, or may be formed as a closed-ended tube. Such an alternate hybrid stopper may provide both piercible access to the interior chamber 20, as well as the venting element for effective displacement of air during sampling and transfer of a sample into tube 12.

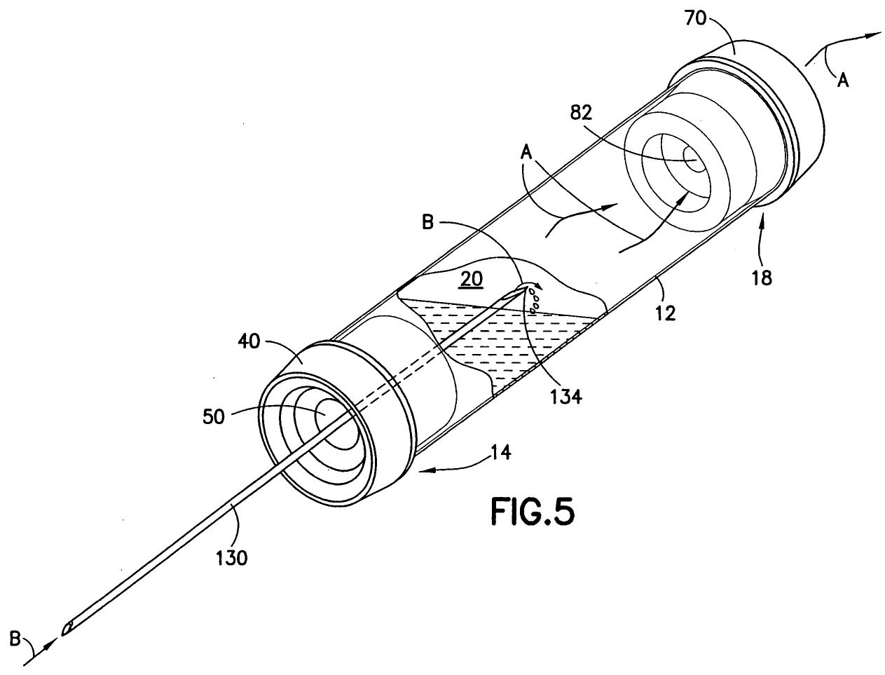

Use of the specimen collection container 10 in connection with drawing a blood sample from a patient is shown in FIG. 5, with cap elements 56 and 86 deleted from FIG. 5 for clarity. In use, a double-ended phlebotomy-type needle such as needle 130 having an intravenous puncture tip (not seen) and a non-patient puncture tip 134 may be connected with a standard needle holder (not shown) and inserted into a patient's blood vessel such as a vein, as is known in the art. In typical blood draw procedures, at this point once the vein is accessed, the non-patient needle 134 is used to pierce a stopper of an evacuated collection tube, and the negative pressure established by the vacuum within such a collection tube draws the blood sample from the patient's vein through the needle cannula and into the tube. As discussed above, however, such procedures can be problematic, with the vacuum oftentimes causing the vein to collapse, particularly for patient's with weakened blood vessels such as infants or the elderly. Also, the exact vacuum within such collection tubes can be difficult to control over time, resulting in a loss of negative pressure within the evacuated tube and an ineffective blood draw.

In the present invention, the non-patient puncture tip 134 of the needle 130 is used to puncture piercible closure 40 through recess 50 of piercible closure 40 at first end 16 of tube 12. As illustrated in FIG. 5, blood travels through needle 130 as shown through arrows B, and enters the interior chamber 20 of tube 12 by way of the patient's blood pressure. More particularly, the blood within the patient's vein is pumping through the vein at physiological pressure, which is greater than normal atmospheric or ambient pressure. Since the interior chamber 20 of tube 12 is vented to the external environment, the air pressure within interior chamber 20 is ambient pressure. When the blood within the patient's vein is in fluid contact with the interior chamber such as when the non-patient puncture tip 134 pierces piercible closure 40, the higher pressure of the patient's vein forces the blood into the lower ambient pressure interior chamber 20. As such, the blood transfer occurs based on the patient's venous pressure.

During such transfer of blood into the interior chamber 20, it is necessary to displace the air within the interior chamber 20 in order to prevent the pressure therein from building to a pressure which would be above the venous pressure and therefore prevent further blood flow into interior chamber 20. Accordingly, during such blood flow, the air, and any other gases present within interior chamber 20, must be displaced from within interior chamber 20. This displacement occurs by venting such air from within interior chamber 20 through venting filter 82 at second end 18 of tube 12, as shown through the directional arrows A. Accordingly, as blood flows into interior chamber 20 through needle 130 as depicted in directional arrows B, the air within interior chamber 20 is correspondingly displaced and passes through venting filter 82 as shown through arrows A. When a sufficient amount of blood is collected within the interior chamber 20 of tube 12, the tube 12 can be removed from non-patient puncture tip 134, thereby stopping the flow of blood into the interior chamber 20.

Also, the nature of venting filter 82 provides a further mechanism for stopping the blood flow. In particular, as noted above, venting filter 82 may be constructed of a material which becomes gas impermeable once it is contacted by blood. During a blood sampling procedure, the container 10 is typically inverted so as to push the piercible closure 40 onto non-patient puncture tip 134. In this position, the second end 18 of tube 12 with venting filter 82 contained within hybrid stopper 70 is positioned at the top of the container 10. Accordingly, during sampling, the blood will rise within the interior chamber 20 of the tube 12 until it is full, at which point the blood will contact the venting filter 82. Such contact will cause venting filter 82 to become gas impermeable, thereby preventing any further blood to enter interior chamber 20 since no further air can be displaced therefrom. Also, such feature serves to protect the blood sample within the tube 12 when a two-way filter is used, since no further air will be able to enter into the interior chamber through the now-sealed venting filter 82 either.

If desired, collection container 10 may be provided with an additive for imparting desired properties to the sample collected therein. For example, one or more additives such as reagents, preservatives, anticoagulants, procoagulants, clot activators, and other known additives may be provided within the interior chamber 20 of tube 12 to provide for a desired effect on the sample. Desirably, such additives are provided in dry form such as a powder or pellet, so as not to effect the gas permeability of venting filter 82.

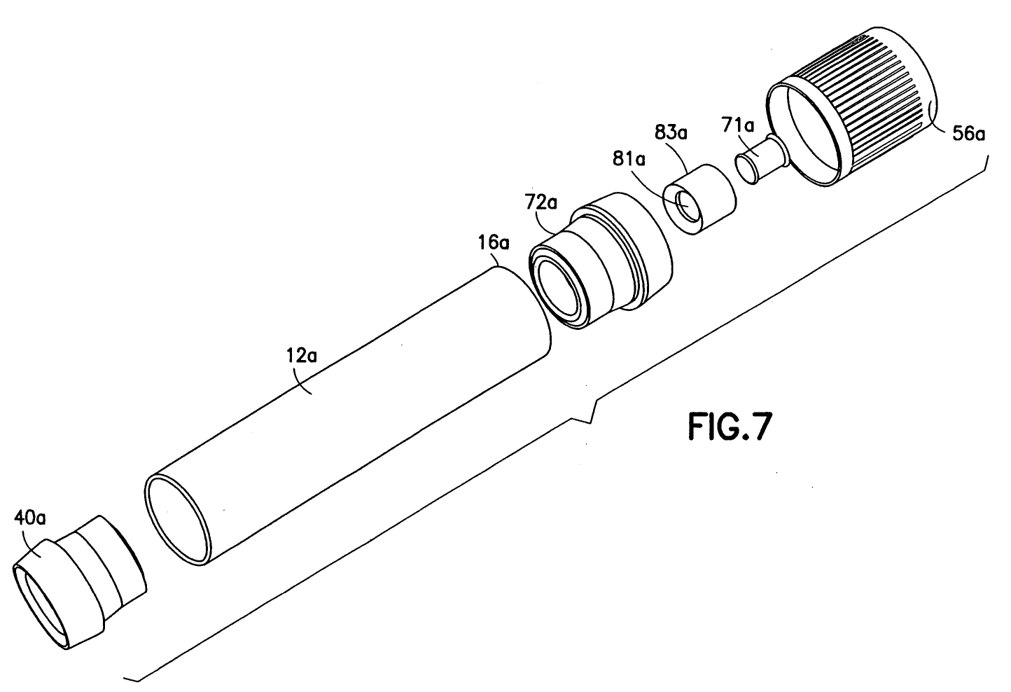

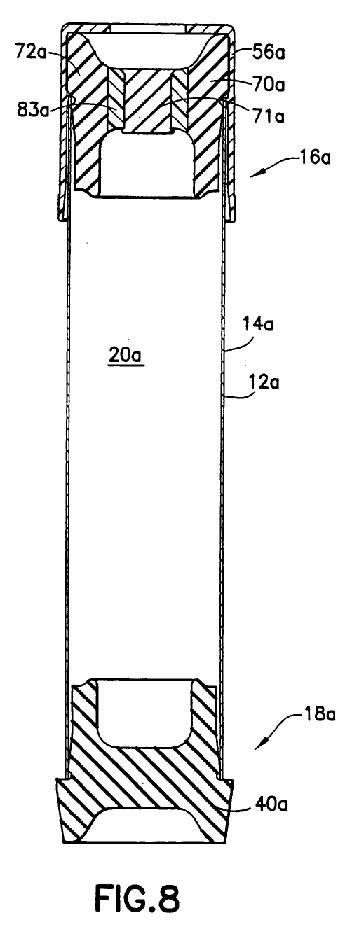

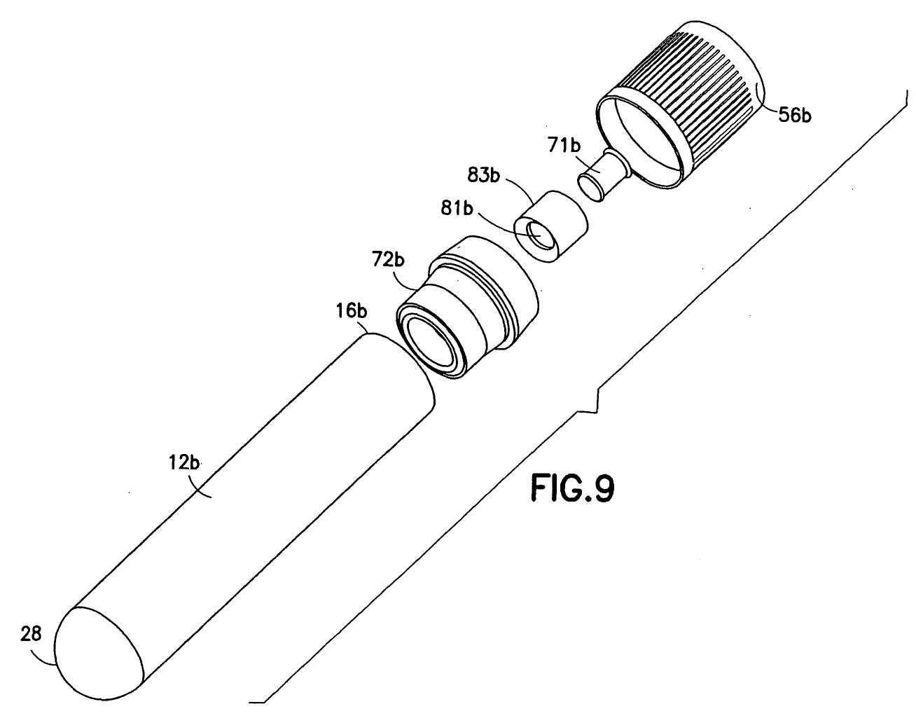

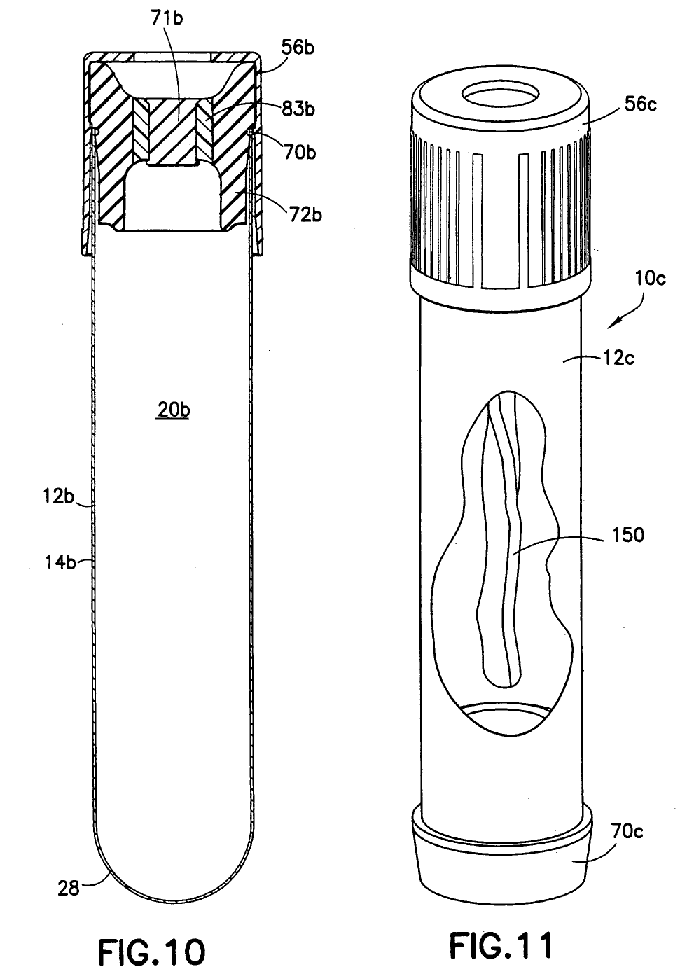

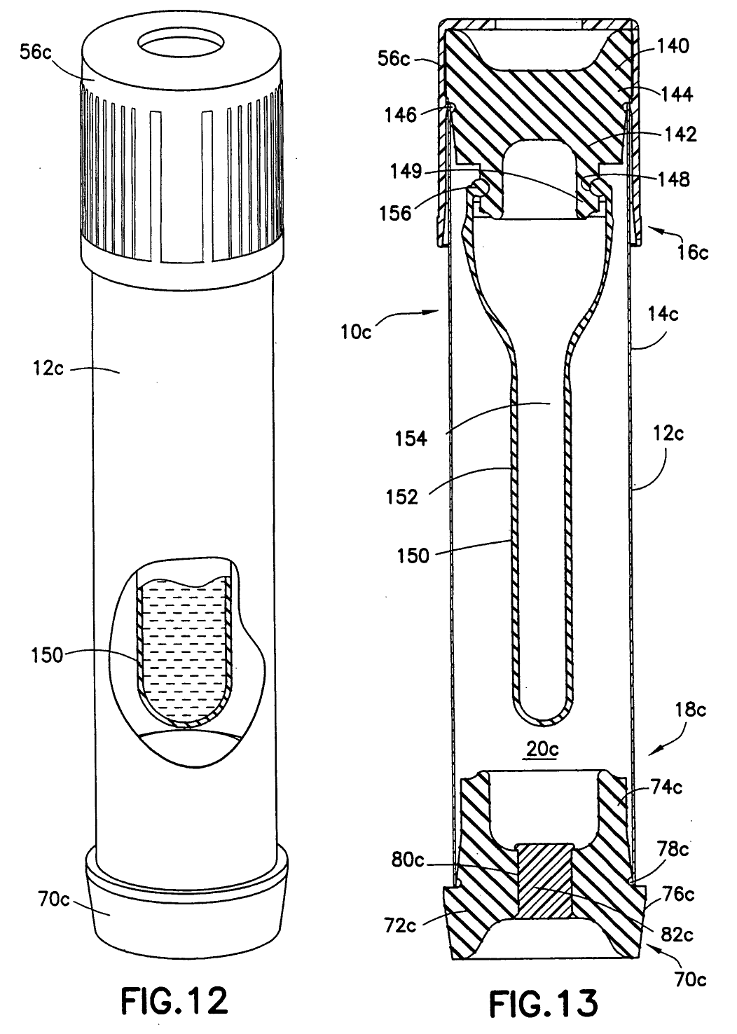

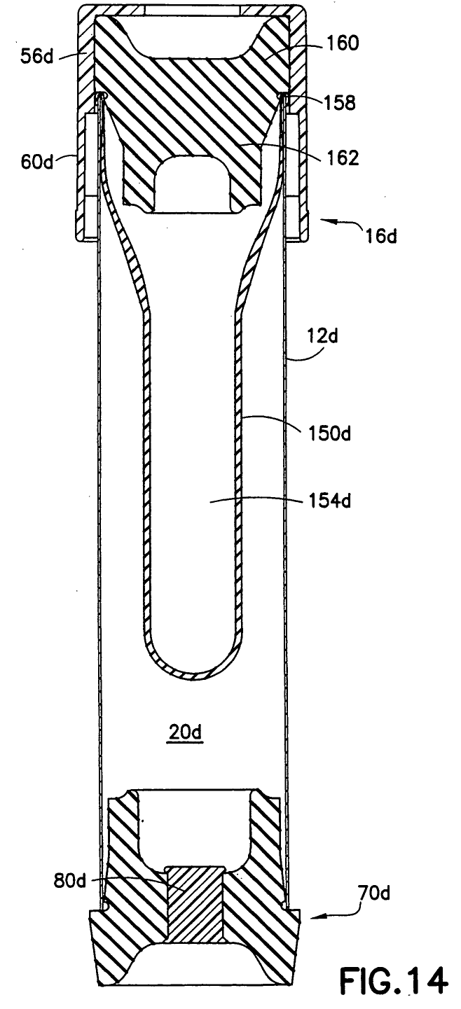

FIGS. 6-14 depict further embodiments of the invention that include many components which are substantially identical to the components of FIGS. 1-4. Accordingly, similar components performing similar functions will be numbered identically to those components of FIGS. 1-4, except that a suffix “a” will be used to identify those similar components in FIGS. 6-8, a suffix “b” will be used to identify those similar components in FIGS. 9-10, a suffix “c” will be used to identify those similar components in FIGS. 11-13, and a suffix “d” will be used to identify those similar components in FIG. 14.

FIGS. 6-8 depict a slight variation on the embodiment of the specimen collection container 10 shown in FIGS. 1-4. In essence, in the embodiment of FIGS. 6-8, the collection container is inverted from that of the previously described embodiment in FIGS. 1-4, such that the hybrid stopper 70a is positioned at the first end 16a of tube 12a. A separate closure 40a is provided at opposing second end 18a. While closure 40a in this embodiment is desirably an elastomeric material and is similar to piercible closure 40 described above, closure 40a is not necessarily intended to be pierced as in the previous embodiment. Instead, in the embodiment of FIGS. 6-8, both the vent means and the means for accessing the interior portion of the collection container are desirably provided through the hybrid stopper 70a. As such, access to interior chamber 20a may be provided through hybrid stopper 70a provided at first end 16a.

More particularly, hybrid stopper 70a may include a specified portion which is adapted for piercing with a needle and a portion which is adapted for venting air therethrough. This may be achieved by providing hybrid stopper 70a with multiple components including elastomeric body 72a having a central bore extending therethrough and an annular venting filter 83a positioned and maintained within the central bore through elastomeric body 72a. Annular venting filter 83a includes a central bore 81a, and an elastomeric plug 71a is further provided and maintained within central bore 81a. In practice of the embodiment shown in FIGS. 6-8, the elastomeric plug 71a is pierced with the non-patient end of a needle for use thereof. Elastomeric plug 71a should be sufficiently maintained within central bore 81a so as not to be displaced during piercing with a needle in such a procedure. This may be accomplished, for example, through friction fit, through the design profile of elastomeric plug 71a interfitting within central bore 81a, or through the use of an adhesive. As blood enters interior chamber 20a of tube 12a, displaced air is forced out of interior chamber 20a through venting filter 83a located within hybrid stopper 70a. Alternatively, elastomeric body 72a of hybrid stopper 70a may be directly pierced with a needle. Cap element 56a may also be provided over first end 16a of tube 12a, thereby encompassing and containing hybrid stopper 70a therein. A separate cap element (not shown in FIGS. 6-8) may be provided over closure 40a at second end 18a if desired.

In yet a further embodiment, the venting filter may be constructed of a material which, in addition to providing the venting and sealing features as described above, is also capable of being pierced with a needle and capable of re-sealing after removal of the needle therefrom. With such an embodiment, the venting filter may be positioned centrally within the stopper element, or may comprise the entire stopper element sealing the end of the tube.

As shown in FIGS. 9-10, a further embodiment of a specimen collection container in accordance with the present invention includes a non-evacuated collection tube 12b having a tubular wall 14b extending between an open top first end 16b and a closed bottom second end 28 defining an interior chamber 20b. In such an embodiment, hybrid stopper 70b including venting filter 83b within elastomeric body 72b is provided at the open top first end 16b of tube 12b, as with the embodiment described in FIGS. 6-8. Such an embodiment provides for a similar collection container as in the embodiment of FIGS. 6-8 and is used in a similar manner, with the exception that the closed second end 28 of tube 12b provides for an entirely closed environment, and eliminates the potential for leaks or spilling through a separate closure such as closure 40a shown in FIGS. 6-8.

FIGS. 11-13 illustrate a further embodiment, which includes a secondary sample container contained within the primary container provided through the specimen collection container, such as an expandable sample bag within the collection container. More particularly, specimen collection container 10c includes a tube 12c, having a closure 140, a hybrid stopper 70c, and an expandable bag 150. Tube 12c includes a tubular wall 14c extending between a first end 16c and a second end 18c, which defines an interior chamber 20c in a similar manner as in the embodiment of FIGS. 1-4. Further, collection container 10c includes a hybrid stopper 70c desirably constructed of an elastomeric body 72c with a depending portion 74c extending within second end 18c of tube 12c, and with annular shoulder 78c resting upon second end 18c. Hybrid stopper 70c also has a central opening 80c medially located with a venting filter 82c located within central opening 80c, as described above.

In the embodiment of FIGS. 11-13, the first end 16c of tube 12c is desirably sealed through closure 140, which may further include a cap element 56c which may be similar in design and construction as that described in the previous emboidments. Closure 140 is mated with the first end 16c of tube 12c through any mechanism, such as a snap-fit, a friction fit, a threaded connection, or the like, with cap element 56c positioned thereover. Closure 140 includes a depending portion 142 extending from a main body portion 144, with depending portion 142 extending within the first end 16c of tube 12c. Closure 140 includes an annular shoulder 146 which mates with first end 16c of tube 12c. The outer diameter of depending portion 142 of piercible closure 140 is substantially the same as the inner diameter of interior chamber 20c of tube 12c. Also, the outer diameter of main portion 144 at annular shoulder 146 of piercible closure 140 is greater than the inner diameter of interior chamber 20c of tube 12c. In this manner, when piercible closure 140 is removably mated to first end 16c of tube 12c annular shoulder 146 rests on the edge of first end 16c, and a gas- and liquid-tight seal is formed. Further, depending portion 142 includes an annular groove 148 extending about a perimeter thereof contained within interior chamber 20c when closure 140 is mated with tube 12c. This annular groove 148 is desirably formed in a narrowed neck portion 149, which has an outer diameter smaller than that of the depending portion 142.

Collection container 10c also includes further structure maintained within interior chamber 20c for holding the blood or biological sample therein. For example, an internal container such as a balloon or an expandable bag 150 may be provided extending into interior chamber 20c. Such expandable bag 150 is adapted for containing the blood or other biological sample entirely therein during the collection procedure, and in effect provides for a secondary container structure for collection of the sample. Expandable bag 150 has a bag wall 152 defining an interior bag chamber 154. Expandable bag 150 may be constructed of any material capable of containing a biological sample and capable of expanding upon exposure to venous pressure. Desirably, expandable bag 150 is a polymeric material which is compatible with blood, has minimal rigidity, and can expand under venous pressure. Examples of useful materials include, but are not limited to, polyethylene or polyvinyl chloride.

Expandable bag extends from the first end 16c within internal chamber 20c of tube 12c. Desirably, expandable bag is mated with closure 140. For example, expandable bag 150 may include an annular ring 156 which is matable with the annular groove 148 on the depending portion 142 of closure 140. In such instances, it may be desirable to include a non-puncturable material at the top portion of expandable bag 150 which is adjacent first end 16c, so as to prevent expandable bag 150 from being punctured by a needle during a sample procedure. By providing expandable bag 150 attached with closure 140, expandable bag 150 with a sample contained therein can be removed from tube 12c along with closure 140 after a sampling procedure in order to access the sample contained within expandable bag 150.

Prior to use, expandable bag 150 is collapsed, with no air contained within interior chamber 154 of expandable bag 150, as shown in FIG. 11. During use, a non-patient puncture tip of a needle can piercably engage closure 140. Such piercing establishes a path for fluid flow through the needle into interior chamber 154 of expandable bag 150. Blood can then enter and fill interior bag chamber 154 of expandable bag 150 based upon the venous pressure, and expandable bag 150 will then expand based upon the blood entering expandable bag 150 under the venous pressure. Expansion of expandable bag 150 will force any air which is present inside interior chamber 20c of tube 12c out to ambient through venting filter 82c at second end 18c of tube 12c. This further embodiment significantly minimizes operator error in that, assuming complete structural integrity of the expandable bag 150 is maintained, blood collected within interior bag chamber 154 of expandable bag 150 will not prematurely contact and seal off venting filter 82c at second end 18c of tube 12c.

FIG. 14 depicts a slight variation of the embodiment in FIGS. 11-13, in which the top end of the expandable bag 150d includes an annular lip portion 158, which is folded over the top edge of first top end 16d of tube 12d (as opposed to the annular ring 156 within the annular groove 148) and is secured in place through frictional engagement on the exterior of first end 16d of tube 12d with annular skirt 60d of cap element 56d and on the interior of first end 16d of tube 12d with closure 160. In this embodiment, the depending portion 162 of closure 160 is substantially smaller in diameter than the inner diameter of tube 12d at first end 16d such that the expandable bag 150d can extend between closure 160 and tube 12d.

While the present invention is satisfied by embodiments in many different forms, there is shown in the figures and described herein in detail, the preferred embodiments of the invention, with the understanding that the present disclosure is to be considered as exemplary of the principles of the invention and is not intended to limit the invention to the embodiments illustrated. Various other embodiments will be apparent to and readily made by those skilled in the art without departing from the scope and spirit of the invention. The scope of the invention will be measured by the appended claims and their equivalents.

1. A specimen collection container comprising:
a non-evacuated collection tube including a tubular wall extending between an open top end and a bottom end defining an interior chamber,
a piercible closure sealing said open top end of said collection tube, and
a vent adapted for displacement of air from said interior chamber of said collection tube to an exterior of said collection tube during collection of a liquid sample within said interior chamber of said collection tube.

2. A specimen collection container as in claim 1, wherein said vent is adapted for maintaining said interior chamber at ambient pressure prior to collection of a sample within said interior chamber.

3. A specimen collection container as in claim 2, wherein said vent comprises a two-way vent.

4. A specimen collection container as in claim 1, wherein said vent is adapted to seal upon contact with said liquid sample so as to prevent said displacement of air.

5. A specimen collection container as in claim 1, wherein said bottom end of said collection container is open, said specimen collection container further comprising a bottom closure sealing said open bottom end of said collection tube with said vent extending through at least a portion of said bottom closure.

6. A specimen collection container as in claim 5, wherein said top closure is removably attached to said open top end of said collection tube and said bottom closure is removably attached to said bottom end of said collection tube.

7. A specimen collection container as in claim 5, wherein said bottom closure comprises a hybrid stopper removably attachable to said open bottom end.

8. A specimen collection container as in claim 7, wherein said hybrid stopper is comprised of an elastomeric material and a venting filter.

9. A specimen collection container as in claim 8, wherein said venting filter comprises a material selected from the group consisting of high density polyethylene and high density polypropylene.

10. A specimen collection container as in claim 1, wherein said vent extends through at least a portion of said piercible closure.

11. A specimen collection container as in claim 10, wherein said piercible closure comprises a hybrid stopper removably attachable to said open top end.

12. A specimen collection container as in claim 10, wherein said hybrid stopper is comprised of an elastomeric material and a venting filter.

13. A specimen collection container as in claim 12, wherein said venting filter comprises a material selected from the group consisting of high density polyethylene and high density polypropylene.

14. A specimen collection container as in claim 1, wherein an expandable bag is positioned in the interior chamber of said tube.

15. A specimen collection container as in claim 14, wherein the expandable bag is mated with said open top end of said collection tube.

16. A specimen collection container as in claim 14, wherein the expandable bag is mated with said piercible closure.

17. A specimen collection container comprising:
a collection container comprising a non-evacuated collection tube including a cylindrical tubular wall extending between an open top end and an open bottom end defining an interior chamber therebetween,
a piercible closure sealing said open top end of said collection tube, and
a venting closure sealing said open bottom end of said collection tube, said venting closure adapted for displacement of air from said interior chamber of said collection tube to an exterior of said collection tube during collection of a liquid sample within said interior chamber of said collection tube.

18. A specimen collection container as in claim 17, wherein said venting closure includes a two-way vent structure.

19. A specimen collection container as in claim 1, wherein said vent is adapted to seal upon contact with said liquid sample so as to prevent said displacement of air.

20. A specimen collection container as in claim 17, wherein said venting closure comprises a hybrid stopper including an elastomeric material containing a venting filter.

21. A specimen collection container as in claim 20, wherein said venting filter comprises a material selected from the group consisting of high density polyethylene and high density polypropylene.

22. A specimen collection container as in claim 17, wherein an expandable bag is positioned in the interior chamber of said tube.

23. A specimen collection container as in claim 22, wherein the expandable bag is mated with said open top end of said collection tube.

24. A specimen collection container as in claim 23, wherein the expandable bag is mated with said piercible closure.

25. A specimen collection container comprising:
a non-evacuated collection tube including a tubular wall extending between an open top end and a bottom end defining an interior chamber,
a piercible closure sealing said open top end of said collection tube,
an internal container positioned within the interior chamber of said collection tube and in sealed engagement with said open top end of said collection tube, said internal container establishing a closed environment for containment of a liquid sample, and
a vent adapted for displacement of air from said interior chamber of said collection tube to an exterior of said collection tube during collection of a liquid sample within said internal container within said interior chamber of said collection tube.

26. A specimen collection container as in claim 25, wherein said internal container comprises an expandable bag positioned in the interior chamber of said tube.

27. A specimen collection container as in claim 26, wherein the expandable bag is mated with said open top end of said collection tube.

28. A specimen collection container as in claim 26, wherein the expandable bag extends from said piercible closure.

29. A specimen collection container as in claim 25, wherein said vent is adapted for maintaining said interior chamber at ambient pressure prior to collection of a sample within said interior chamber.

30. A specimen collection container as in claim 29, wherein said vent comprises a two-way vent.

31. A specimen collection container as in claim 25, wherein said vent is adapted to seal upon contact with said liquid sample so as to prevent said displacement of air.

32. A specimen collection container as in claim 25, wherein said bottom end of said collection container is open, said specimen collection container further comprising a bottom closure sealing said open bottom end of said collection tube with said vent extending through at least a portion of said bottom closure.

33. A specimen collection container as in claim 32, wherein said top closure is removably attached to said open top end of said collection tube and said bottom closure is removably attached to said bottom end of said collection tube.

34. A specimen collection container as in claim 32, wherein said bottom closure comprises a hybrid stopper removably attachable to said open bottom end.

35. A specimen collection container as in claim 34, wherein said hybrid stopper is comprised of an elastomeric material and a venting filter.

36. A specimen collection container as in claim 35, wherein said venting filter comprises a material selected from the group consisting of high density polyethylene and high density polypropylene.

37. A method for collecting a biological sample comprising the steps of:
a) providing a non-evacuated collection tube including a vent adapted for displacement of air from within said collection tube to an exterior environment and a piercible closure providing access to the interior of said collection tube;
b) accessing the interior of said collection tube through said piercible closure; and
c) transferring a biological sample through said piercible closure into said interior of said collection tube such that any air present within said interior of said collection tube will vent to the exterior environment through said vent during such transferring step.

38. The method of claim 37, wherein said accessing step comprises establishing fluid communication between a blood vessel of a patient and the interior of said collection tube, such that blood pressure causes transferring of blood from the blood vessel to the interior of the collection tube.

39. The method of claim 38, wherein said vent is adapted to seal upon contact with said biological sample so as to prevent said displacement of air.

40. The method of claim 38, wherein said vent is protected from said biological sample by the presence of an expandable bag positioned in the interior chamber of said tube.
